(19) United States
(12) Patent Application Publication
(10) Pub. No.: US 2010/0298870 A1
(43) Pub. Date: Nov. 25, 2010

(54) ABSORBABLE SURGICAL FASTENERS

(75) Inventors: Ahmad R. Hadba, Wallingford, CT (US); Walter Skalla, Old Lyme, CT (US)

Correspondence Address: Tyco Healthcare Group LP;d/b/a Covidien, 555 Long Wharf Drive, Mail Stop 8-N1, Legal Department, New Haven, CT, 06511, US

(73) Assignee: TYCO HEALTHCARE GROUP LP, New Haven, CT (US)

(21) Appl. No.: 12/848,691

(22) Filed: Aug. 2, 2010

Related U.S. Application Data
(63) Continuation of application No. 11/885,888, filed on Sep. 6, 2007.

(30) Foreign Application Priority Data
Mar. 10, 2006 (US) PCT/US06/08759

(51) Int. Cl. A61B 17/064

(52) U.S. Cl. 606/219

(57) ABSTRACT

The present disclosure provides absorbable surgical fasteners.

### CROSS REFERENCE TO RELATED APPLICATIONS

The present application is a continuation application claiming the benefit of and priority to U.S. application Ser. No. 11/885,888 filed on Sep. 6, 2007, which is a National Phase Application filed under 35 U.S.C. §371 of International Application Serial No. PCT/US2006/008759, filed Mar. 10, 2006, which claims the benefit of and priority to U.S. Provisional Application Ser. No. 60/660,694 filed on Mar. 11, 2005, the entire content of each of these Applications is incorporated herein by reference.

### BACKGROUND

1. Technical Field

The present disclosure relates to absorbable surgical fasteners (e.g., staples) and more particularly to absorbable metallic surgical fasteners.

2. Background of Related Art

Surgical fastening devices allow a surgeon to connect body tissue by applying surgical fasteners. The fasteners may be applied singly in succession or any number may be applied simultaneously. Surgical fasteners are known to be made by metallic and non-metallic materials.

Metallic fasteners are often made of materials such as tantalum or stainless steel, which are inert. However, metallic fasteners are not typically absorbable and therefore are frequently permanently implanted or are used on wound sites that allow the metallic fastener to be removed from the wound site after the wound has sufficiently healed.

Non-metallic fasteners are usually absorbable and often made from materials such as natural or synthetic polymers or copolymers and resins, including protein based-materials. However, non-metallic fasteners often experience difficulty retaining their tensile strength for a time sufficient to allow the fastened tissue to heal. Also, they often are not stiff enough to penetrate certain tissue without the assistance from metallic pins, drills and the like.

Thus, the need exists for absorbable metallic surgical fasteners that maintain sufficient stiffness to penetrate tissue, maintain sufficient tensile strength during the healing process and produce no harmful effects to the body when absorbed.

### SUMMARY

The present disclosure provides a surgical fastener for closing wounds and a method of using the fastener. The surgical fastener comprises a combination of metal materials that dissolve in a human body without any harmful effects. The method of using the fastener includes approximating the tissue surrounding the wound and affixing a surgical fastener described herein to the approximated tissue.

### DETAILED DESCRIPTION

The present disclosure provides a surgical fastener comprising a combination of metal materials which dissolve in the human body without any harmful effects on the person who wears the implant. The combination of metal materials is to be designed such that the material of the surgical fastener dissolves at a certain decomposition rate and without the production of bio-incompatible decomposition products. A surgical fastener of this type combines the advantageous mechanical properties of metallic surgical fasteners with the bioabsorbability of non-metallic, or polymer-based surgical fasteners.

In one embodiment, the combination of metal materials is a metal alloy, the selection of the alloy constituents—as explained in detail below—serving to attain the prerequisite of biocompatible decomposition. Consequently, the metal alloy has to consist of a combination of material that will dissolve in the body comparatively rapidly—within a period of some months—forming harmless constituents.

For correspondingly uniform decomposition to be obtained, such an alloy comprises a first component which covers itself with a protective oxide coat. This first component is selected from one or several metals of the group of magnesium, titanium, zirconium, niobium, tantalum, zinc or silicon. For uniform dissolution of the mentioned oxide coat to be attained, a second component is added to the alloy, possessing sufficient solubility in blood or interstitial fluid, such as lithium sodium, potassium, calcium, iron or manganese.

The mentioned elements are suitable because they are present in the human body anyway—such as magnesium, zinc, sodium, potassium, calcium, iron and manganese—or are known to be nontoxic—such as titanium, zirconium, niobium, tantalum, silicon and lithium. The combination of a passivating and a soluble component ensures a timely and uniform decomposition into biocompatible breakdown products. The decomposition rate can be regulated through the ratio of the two components.

In a particularly useful embodiment, the alloy is to be formed so that the decomposition products are soluble salts, such as sodium, potassium, calcium, iron or zinc salts, or that non-soluble decomposition products, such as titanium, tantalum or niobium oxide originate as colloidal particles. The decomposition rate is adjusted by way of the composition so that gases, such as hydrogen which evolves during the decomposition of lithium, sodium, potassium, magnesium, calcium or zinc, dissolve physically, not forming any macroscopic gas bubbles.

One combination of alloys that is particularly useful is a sodium-magnesium alloy. Since sodium hydroxide as a decomposition product possesses a high solubility, this alloy decomposes without voluminous encrusting. Sodium dissolves and magnesium hydroxide forms a fine precipitate which may deposit without risk in the wound healing tissue.

Another useful decomposable combination of metal materials is a zinc-titanium alloy, the percentage by weight of which is in the range of 0.1% to 1%. This combination precludes the comparatively strong crystalline growth of zinc as a material used, which would cause a comparatively brittle and fragile behavior of the surgical fastener. When the material is worked, the addition of titanium leads to the formation of a Zn₁₅Ti phase on the crystal boundaries which precludes any further crystalline growth. This reduction of the grain size generally improves the ductility, in particular the elongation at rupture—i.e. the percentage elongation of the material under mechanical load as far as to the rupture thereof.

If gold is added to this alloy at a percentage by weight of 0.1% to 2%, a further reduction of the grain size is attained when the material cures. This further improves the tensile strength of the material.

The surgical fasteners of the present disclosure can be used to close a wound on skin, fascia or internal organs. The closure of a wound involves the approximating, abutting, and/or overlapping of tissue surrounding the wound and placing the fastener in a position relative to the wound to maintain the surrounding tissue in the approximated, abutted and/or overlapped position until the wound has healed.

Any surgical fastener known to one skilled in the art may be formed from the metallic alloy described in the present disclosure. Additionally, the surgical fasteners can be formed into any shape, size and dimension useful in closing wounds. Some examples of surgical fasteners include staples, pins, straps, cables, screws and clips. In a particularly useful embodiment the fastener is a surgical staple.

In the case of staples, a wide variety of surgical staples and surgical staplers are known and used throughout the art. The surgical staples described herein can be adapted for use with any surgical staplers of a conventional design. Examples of such devices are described in U.S. Pat. Nos. 4,354,628, 5,014,899, 5,040,715, 5,799,857 and 5,915,616. The staples can be used in cartridge fed, repeating stapling instruments or in instruments which set a plurality of staples in a straight line or in a circle with a single firing is also included within the scope of this invention. It is understood that some modification of existing stapling instruments may be required to physically accommodate the staples of the present invention, but such modification is well within the present skill of the instrument manufacturers.

It is envisioned that surgical fasteners as described herein would be used in conjunction with other surgically biocompatible wound treatment materials that include, adhesives whose function is to attach or hold organs, tissues or structures; sealants to prevent fluid leakage; hemostats to halt or prevent bleeding; and medicaments. Examples of adhesives which can be employed include protein derived, aldehyde-based adhesive materials, for example, the commercially available albumin/glutaraldehyde materials sold under the trade designation BioGlue™ by Cryolife, Inc., and cyanoacrylate-based materials sold under the trade designations Indermil™ and Derma Bond™ by Tyco Healthcare Group, LP and Ethicon Endosurgery, Inc., respectively. Examples of sealants, which can be employed, include fibrin sealants and collagen-based and synthetic polymer-based tissue sealants. Examples of commercially available sealants are synthetic polyethylene glycol-based, hydrogel materials sold under the trade designation CoSeal™ by Cohesion Technologies and Baxter International, Inc. Examples of hemostat materials, which can be employed, include fibrin-based, collagen-based, oxidized regenerated cellulose-based and gelatin-based topical hemostats. Examples of commercially available hemostat materials are fibrinogen-thrombin combination materials sold under the trade designations CoStasis™ by Tyco Healthcare Group, LP, and Tisseel™ sold by Baxter International, Inc. Hemostats herein include astringents, e.g., aluminum sulfate, and coagulants.

The adhesive, sealant or medicament may be disposed on or impregnated into any of the surgical fasteners described herein. The medicament may include one or more medically and/or surgically useful substances such as drugs, enzymes, growth factors, peptides, proteins, dyes, diagnostic agents or hemostasis agents or any other pharmaceutical used in the prevention of stenosis.

Obviously many modifications and variations of the present invention are possible in light of the above teachings and it is contemplated that, within the scope of the appended claims, the invention may be practiced otherwise than as specifically described.

What is claimed is:

1. A surgical fastener comprising:
a staple made from a metal alloy that dissolves in a human body without any harmful effects on the wearer of the surgical fastener, the metal alloy comprising a first component and a second component;
wherein the first component includes at least one of titanium, zirconium, nobium, and tantalum and the second component includes at least one of lithium, sodium, potassium, manganese, calcium, zinc, and iron.

2. The surgical fastener of claim 1 wherein the first component forms a protecting passivation coat and the second component ensures sufficient corrosion of the alloy.

3. The surgical fastener of claim 1 wherein the first component is titanium.

4. The surgical fastener of claim 1 wherein the first component is zirconium.

5. The surgical fastener of claim 1 wherein the first component is nobium.

6. The surgical fastener of claim 1 wherein the first component is tantalum.

7. The surgical fastener of claim 1 wherein the second component is lithium.

8. The surgical fastener of claim 1 wherein the second component is potassium.

9. The surgical fastener of claim 1 wherein the second component is manganese.

10. The surgical fastener of claim 1 wherein the second component is calcium.

11. The surgical fastener of claim 1 wherein the second component is zinc.

12. The surgical fastener of claim 1 wherein the second component is iron.

13. The surgical fastener of claim 1 further comprising an additional component selected from the group consisting of tissue adhesive, tissue sealant and medicament.

14. The surgical fastener of claim 1 wherein the metal alloy further comprises gold.

15. A method of closing a wound comprising the steps of:
approximating tissue surrounding the wound; and
affixing the surgical fastener of claim 1 to the approximated tissue.

16. A surgical fastener comprising:
a staple made from a zinc-titanium alloy that dissolves in a human body, wherein upon dissolving the alloy forms a soluble zinc salt and colloidal particles of titanium.

17. The surgical fastener of claim 16 further comprising an additional component selected from the group consisting of tissue adhesive, tissue sealant and medicament.

18. The surgical fastener of claim 16 wherein the metal alloy further comprises gold.
